SEQUENCE LISTING (57 sequences)

SEQ ID NO: 1 - protein, 133 aa, Homo sapiens [sequence omitted]

SEQ ID NO: 2 - protein, 34 aa, synthetic construct
APTSSSTKKTQLQLEHLLLDLQMILNGINNYKNP

SEQ ID NO: 3 - protein, 34 aa, synthetic construct
APTSSSTKKTCLQLEHLLLDLQMILNGINNYKNP

SEQ ID NO: 4 - protein, 34 aa, synthetic construct
APTSSSTKKTQLQLEHCLLDLQMILNGINNYKNP

SEQ ID NO: 5 - protein, 34 aa, synthetic construct
APTSSSTKKTQLQLEHLCLDLQMILNGINNYKNP

SEQ ID NO: 6 - protein, 34 aa, synthetic construct
APTSSSTKKTQLQLEHLLLDLQMILNGINNCKNP

SEQ ID NO: 7 - protein, 38 aa, synthetic construct
KLTRMLTFKFYMPKKATELKHLQCLEEELKPLEEVLNL

SEQ ID NO: 8 - protein, 38 aa, synthetic construct
KLTRMLTFCFYMPKKATELKHLQCLEEELKPLEEVLNL

SEQ ID NO: 9 - protein, 38 aa, synthetic construct
KLTRMLTFKCYMPKKATELKHLQCLEEELKPLEEVLNL

SEQ ID NO: 10 - protein, 38 aa, synthetic construct
KLTRMLTFKFYMPKKATELKHCQCLEEELKPLEEVLNL

SEQ ID NO: 11 - protein, 61 aa, synthetic construct
AQSKNFHLRPRDLISNINVIVLELKGSETTFMCEYADETATIVEFLNRWITFCQSIISTLT

SEQ ID NO: 12 - protein, 61 aa, synthetic construct
AQSKNFHLRPRDLISNINVIVLELKGSETTFMCEYADETATIVEFLNRWITFSQSIISTLT

SEQ ID NO: 13 - protein, 61 aa, synthetic construct
AQSKNFHLRPRDLISNINVIVLELKGSETTFMCEYADETATIVEFLNRWITFAQSIISTLT

SEQ ID NO: 14 - protein, 61 aa, synthetic construct
AQSKNFHLRPRDLISNINVIVLELKGSETTFMCEYADETATIVEFLNRWITFLQSIISTLT

SEQ ID NO: 15 - protein, 61 aa, synthetic construct
AQSKCFHLRPRDLISNINVIVLELKGSETTFMCEYADETATIVEFLNRWITFSQSIISTLT

SEQ ID NO: 16 - protein, 61 aa, synthetic construct
AQSKCFHLRPRDLISNINVIVLELKGSETTFMCEYADETATIVEFLNRWITFAQSIISTLT

SEQ ID NO: 17 - protein, 61 aa, synthetic construct
AQSKCFHLRPRDLISNINVIVLELKGSETTFMCEYADETATIVEFLNRWITFLQSIISTLT

SEQ ID NO: 18 - protein, 61 aa, synthetic construct
AQSKNFHCRPRCLISNINVIVLELKGSETTFMCEYADETATIVEFLNRWITFSQSIISTLT

SEQ ID NO: 19 - protein, 61 aa, synthetic construct
AQSKNFHCRPRCLISNINVIVLELKGSETTFMCEYADETATIVEFLNRWITFAQSIISTLT

SEQ ID NO: 20 - protein, 61 aa, synthetic construct
AQSKNFHCRPRCLISNINVIVLELKGSETTFMCEYADETATIVEFLNRWITFLQSIISTLT

SEQ ID NO: 21 - protein, 61 aa, synthetic construct
AQSKNFHLRPRDLISNINVCVLELKGSETTFMCEYADETATIVEFLNRWITFSQSIISTLT

SEQ ID NO: 22 - protein, 61 aa, synthetic construct
AQSKNFHLRPRDLISNINVCVLELKGSETTFMCEYADETATIVEFLNRWITFAQSIISTLT

SEQ ID NO: 23 - protein, 61 aa, synthetic construct
AQSKNFHLRPRDLISNINVCVLELKGSETTFMCEYADETATIVEFLNRWITFLQSIISTLT

SEQ ID NO: 24 - protein, 61 aa, synthetic construct
AQSKNFHLRPRDLISNINVIVLELCGSETTFMCEYADETATIVEFLNRWITFSQSIISTLT

SEQ ID NO: 25 - protein, 61 aa, synthetic construct
AQSKNFHLRPRDLISNINVIVLELCGSETTFMCEYADETATIVEFLNRWITFAQSIISTLT

SEQ ID NO: 26 - protein, 61 aa, synthetic construct
AQSKNFHLRPRDLISNINVIVLELCGSETTFMCEYADETATIVEFLNRWITFLQSIISTLT

SEQ ID NO: 27 - protein, 61 aa, synthetic construct
AQSKNFHLRPRDLISNINVIVLELKGSETTFMCEYADECATIVEFLNRWITFSQSIISTLT

SEQ ID NO: 28 - protein, 61 aa, synthetic construct
AQSKNFHLRPRDLISNINVIVLELKGSETTFMCEYADECATIVEFLNRWITFAQSIISTLT

SEQ ID NO: 29 - protein, 61 aa, synthetic construct
AQSKNFHLRPRDLISNINVIVLELKGSETTFMCEYADECATIVEFLNRWITFLQSIISTLT

SEQ ID NO: 30 - protein, 61 aa, synthetic construct
AQSKNFHLRPRDLISNINVIVLELKGSETTFMCEYADETATIVECLNRWITFSQSIISTLT

SEQ ID NO: 31 - protein, 61 aa, synthetic construct
AQSKNFHLRPRDLISNINVIVLELKGSETTFMCEYADETATIVECLNRWITFAQSIISTLT

SEQ ID NO: 32 - protein, 61 aa, synthetic construct
AQSKNFHLRPRDLISNINVIVLELKGSETTFMCEYADETATIVECLNRWITFLQSIISTLT

SEQ ID NO: 33 - protein, 61 aa, synthetic construct
AQSKNFHLRPRDLISNINVIVLELKGSETTFMCEYADETATIVEFLNRWITFSQSIISTCT

SEQ ID NO: 34 - protein, 61 aa, synthetic construct
AQSKNFHLRPRDLISNINVIVLELKGSETTFMCEYADETATIVEFLNRWITFAQSIISTCT

SEQ ID NO: 35 - protein, 61 aa, synthetic construct
AQSKNFHLRPRDLISNINVIVLELKGSETTFMCEYADETATIVEFLNRWITFLQSIISTCT

SEQ ID NO: 36 - protein, 133 aa, synthetic construct
APTSSSTKKTCLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTCT

SEQ ID NO: 37 - protein, 133 aa, synthetic construct
APTSSSTKKTCLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTCT

SEQ ID NO: 38 - protein, 133 aa, synthetic construct
APTSSSTKKTCLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFLQSIISTCT

SEQ ID NO: 39 - protein, 133 aa, synthetic construct
APTSSSTKKTQLQLEHCLLDLQMILNGINNYKNPKLTRMLTFKFYMPKKATELKHLQCLEEELKPLEEVLNLAQSKNFHLRPRDLISNINVCVLELKGSETTFMCEYADETATIVEFLNRWITFSQSIISTLT

SEQ ID NO: 40 - protein, 133 aa, synthetic construct
APTSSSTKKTQLQLEHCLLDLQMILNGINNYKNPKLTRMLTFKFYMPKKATELKHLQCLEEELKPLEEVLNLAQSKNFHLRPRDLISNINVCVLELKGSETTFMCEYADETATIVEFLNRWITFAQSIISTLT

SEQ ID NO: 41 - protein, 133 aa, synthetic construct
APTSSSTKKTQLQLEHCLLDLQMILNGINNYKNPKLTRMLTFKFYMPKKATELKHLQCLEEELKPLEEVLNLAQSKNFHLRPRDLISNINVCVLELKGSETTFMCEYADETATIVEFLNRWITFLQSIISTLT

SEQ ID NO: 42 - protein, 133 aa, synthetic construct
APTSSSTKKTQLQLEHLCLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 43 - protein, 133 aa, synthetic construct
APTSSSTKKTQLQLEHLLLDLQMILNGINNCKNPKLTRMLTFKFYMPKKATELKHLQCLEEELKPLEEVLNLAQSKCFHLRPRDLISNINVIVLELKGSETTFMCEYADETATIVEFLNRWITFSQSIISTLT

SEQ ID NO: 44 - protein, 133 aa, synthetic construct
APTSSSTKKTQLQLEHLLLDLQMILNGINNCKNPKLTRMLTFKFYMPKKATELKHLQCLEEELKPLEEVLNLAQSKCFHLRPRDLISNINVIVLELKGSETTFMCEYADETATIVEFLNRWITFAQSIISTLT

SEQ ID NO: 45 - protein, 133 aa, synthetic construct
APTSSSTKKTQLQLEHLLLDLQMILNGINNCKNPKLTRMLTFKFYMPKKATELKHLQCLEEELKPLEEVLNLAQSKCFHLRPRDLISNINVIVLELKGSETTFMCEYADETATIVEFLNRWITFLQSIISTLT

SEQ ID NO: 46 - protein, 133 aa, synthetic construct
APTSSSTKKTQLQLEHLLLDLQMILNGINNYKNPKLTRMLTFCFYMPKKATELKHLQCLEEELKPLEEVLNLAQSKNFHLRPRDLISNINVIVLELKGSETTFMCEYADECATIVEFLNRWITFSQSIISTLT

SEQ ID NO: 47 - protein, 133 aa, synthetic construct
APTSSSTKKTQLQLEHLLLDLQMILNGINNYKNPKLTRMLTFCFYMPKKATELKHLQCLEEELKPLEEVLNLAQSKNFHLRPRDLISNINVIVLELKGSETTFMCEYADECATIVEFLNRWITFAQSIISTLT

SEQ ID NO: 48 - protein, 133 aa, synthetic construct
APTSSSTKKTQLQLEHLLLDLQMILNGINNYKNPKLTRMLTFCFYMPKKATELKHLQCLEEELKPLEEVLNLAQSKNFHLRPRDLISNINVIVLELKGSETTFMCEYADECATIVEFLNRWITFLQSIISTLT

SEQ ID NO: 49 - protein, 133 aa, synthetic construct
APTSSSTKKTQLQLEHLLLDLQMILNGINNYKNPKLTRMLTFKCYMPKKATELKHLQCLEEELKPLEEVLNLAQSKNFHLRPRDLISNINVIVLELKGSETTFMCEYADETATIVECLNRWITFSQSIISTLT

SEQ ID NO: 50 - protein, 133 aa, synthetic construct
APTSSSTKKTQLQLEHLLLDLQMILNGINNYKNPKLTRMLTFKCYMPKKATELKHLQCLEEELKPLEEVLNLAQSKNFHLRPRDLISNINVIVLELKGSETTFMCEYADETATIVECLNRWITFAQSIISTLT

SEQ ID NO: 51 - protein, 133 aa, synthetic construct
APTSSSTKKTQLQLEHLLLDLQMILNGINNYKNPKLTRMLTFKCYMPKKATELKHLQCLEEELKPLEEVLNLAQSKNFHLRPRDLISNINVIVLELKGSETTFMCEYADETATIVECLNRWITFLQSIISTLT

SEQ ID NO: 52 - protein, 133 aa, synthetic construct
APTSSSTKKTQLQLEHLLLDLQMILNGINNYKNPKLTRMLTFKFYMPKKATELKHCQCLEEELKPLEEVLNLAQSKNFHLRPRDLISNINVIVLELCGSETTFMCEYADETATIVEFLNRWITFSQSIISTLT

SEQ ID NO: 53 - protein, 133 aa, synthetic construct
APTSSSTKKTQLQLEHLLLDLQMILNGINNYKNPKLTRMLTFKFYMPKKATELKHCQCLEEELKPLEEVLNLAQSKNFHLRPRDLISNINVIVLELCGSETTFMCEYADETATIVEFLNRWITFAQSIISTLT

SEQ ID NO: 54 - protein, 133 aa, synthetic construct
APTSSSTKKTQLQLEHLLLDLQMILNGINNYKNPKLTRMLTFKFYMPKKATELKHCQCLEEELKPLEEVLNLAQSKNFHLRPRDLISNINVIVLELCGSETTFMCEYADETATIVEFLNRWITFLQSIISTLT

SEQ ID NO: 55 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHCRPRCLISNINVIVLELKGSETTFMCEYADETATIVEFLNRWITFSQSIISTLT

SEQ ID NO: 56 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHCRPRCLISNINVIVLELKGSETTFMCEYADETATIVEFLNRWITFAQSIISTLT

SEQ ID NO: 57 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHCRPRCLISNINVIVLELKGSETTFMCEYADETATIVEFLNRWITFLQSIISTLT

(19) United States
(12) Patent Application Publication
(10) Pub. No.: US 2026/0167689 A1
(43) Pub. Date: Jun. 18, 2026

(54) POLYPEPTIDE, MEDICINE COMPOSITION INCLUDING POLYPEPTIDE AND USE THEREOF

(71) Applicant: National Cheng Kung University, Tainan (TW)

(72) Inventor: Woei-Jer CHUANG, Tainan (TW)

(21) Appl. No.: 19/020,725

(22) Filed: Jan. 14, 2025

(30) Foreign Application Priority Data
Dec. 12, 2024 (TW) 113148413

(51) Int. Cl. C07K 14/55

(52) U.S. Cl. CPC C07K 14/55

(57) ABSTRACT

The present invention discloses a polypeptide, medicine composition including it and use thereof. The polypeptide comprises: a first structural domain, a second structural domain and a third structural domain, wherein the second structural domain is an interleukin-2 receptor subunit α-binding domain, and the first structural domain and the third structural domain are non-interleukin-2 receptor subunit α-binding domain. A first mutation is introduced into the third structural domain by substitution with cysteine, and a second mutation is introduced optionally into the first structural domain, the second structural domain or the third structural domain by substitution with cysteine, and a disulfide bond forms therebetween. The polypeptide presents better thermal stability, solubility and productivity, and shows tendency to bind IL-2Rα or IL-2Rβγ. The polypeptide is able to activate anti-cancer immune response by regulation of regulatory T cells or effector T cells.

### CROSS-REFERENCE TO RELATED APPLICATION

This application claims the benefit of Taiwan Patent application Ser. No. 113148413, filed Dec. 12, 2024. The disclosure of the above application is incorporated herein in its entirety by reference.

The sequence information contained in the Sequence Listing XML file, with the file name PI-113-198-US-SEQUENCE LISTING-20250107.xml, having a size of 54,863 bytes and being created on Dec. 26, 2024, is incorporated herein by reference in its entirety.

### FIELD OF THE INVENTION

The present invention is related to protein engineering, especially related to 3D structural modification and uses of interleukin-2 and protein derivatives thereof; the uses are related to a method for prevention, relief or cure of a disease or a disorder resulting from immune cells of low activity.

### BACKGROUND OF THE INVENTION

Cancer is the second major cause of death worldwide. Immune therapy attacks cancer cells via the patient's own immune system. Interleukin-2 (IL-2 hereinafter) activates T cells and promote their cell proliferation, and high-dose IL-2 has been the first approved immune therapy against metastatic renal cell carcinoma and melanoma.

Although IL-2 induces durable anti-cancer responses in a small number of patients and extend survival time, it is not without side effects. For instance, it can cause capillary leak syndrome, and the specific pathogenesis remains elusive. On the other hand, persistent activation of IL-2 receptor subunit α may lead to T cells skewing towards CD8⁺ T cells. Furthermore, continuous expansion of regulatory T cells' population will consume T cell reserve and suppresses activity of effector T cells, which may induce immune escape effect and excessive secretion of suppressive cell factors. As such, tumor progression or cytokine storm may eventually result in severe consequences such as multiple organ dysfunction. Therefore, therapeutic plan using IL-2 must be applied with great caution.

In addition, as IL-2 is characterized of low water solubility, poor thermostability and short half-life, it requires multiple-time injections of high dosage in cancer treatment, thereby risking patients of side effects and limiting its therapeutic efficacy.

The primary technique of IL-2 massive production is chemical protein synthesis, and the major product is IL-2 analogue presenting biological activity. By using chemical synthesis, IL-2 mutant forms with concise structures and its non-natural analogue with disulfide bond can be created, but their half-life are still short, only last for 7 minutes. Moreover, water solubility issues of hydrophobic fragment in IL-2 are not yet overcome even by using KAHA ligation technique, thereby limiting IL-2 productivity. On top of that, IL-2 analogues prepared by using current techniques show more tendency of binding to IL-2 receptor subunit α, and activate regulatory T cell (Treg), which inevitably suppresses anti-cancer immune response.

### SUMMARY OF THE INVENTION

The present invention is made based on the following design: a disulfide bond is introduced to structural domains of interleukin-2 so as to obtain a mutant form containing a disulfide bond. Thermal stability, solubility and capability of immune cell activation of such mutant form is overall increased. Additionally, the mutant IL-2 with introduction of the disulfide bond demonstrates differential affinity to IL-2 receptor subunits, and has tendency to either bind to subunit α or subunits βγ, thereby being a potential candidate of biomedicine.

Accordingly, provided in one aspect of the present invention is a polypeptide, comprising: a first structural domain, comprising an amino acid sequence with at least 80%, 85%, 90%, 95% or 99% similarity to an amino acid sequence at position 1 to position 34 of SEQ ID NO: 1; a second structural domain, configured at C-terminus of the first structural domain, comprising an amino acid sequence with at least 80%, 85%, 90%, 95% or 99% similarity to an amino acid sequence at position 35 to position 72 of SEQ ID NO: 1; and a third structural domain, configured at C-terminus of the second structural domain, comprising an amino acid sequence with at least 80%, 85%, 90%, 95% or 99% similarity to an amino acid sequence at position 73 to position 133 of SEQ ID NO: 1, and being introduced of a first mutation by a substitution of a first amino acid residue with a cysteine residue, wherein: the polypeptide is introduced of a second mutation by a substitution of a second amino acid residue with another cysteine residue, and the second mutation optionally happens in the first structural domain, the second structural domain or the third structural domain, thereby forming a disulfide bond between the cysteine residue introduced by the first mutation and the cysteine residue introduced by the second mutation.

Preferably, the first amino acid residue comprises a asparagine residue (Asn, N) at position 77, an aspartate residue (Asp, D) at position 84, a isoleucine residue (Ile, I) at position 92, Lysine residue (Lys, K) at position 97, a threonine residue (Thr, T) at position 111, a phenylalanine residue (Phe, F) at position 117 or a leucine residue (Leu, L) at position 132; the second amino acid residue comprises a glutamine residue (Gln, Q) at position 11, a leucine residue (Leu, L) at position 17, a leucine residue (Leu, L) at position 18, a tyrosine residue (Tyr, Y) at position 31, a lysine residue (Lys, K) at position 43, a phenylalanine residue (Phe, F) at position 44, a leucine residue (Leu, L) at position 56 or a leucine residue (Leu, L) at position 80.

Preferably, the first amino acid residue comprises a threonine residue (Thr, T) at position 111, and the second amino acid residue comprises a lysine residue (Lys, K) at position 43.

Preferably, in C-terminus downstream area of the first amino acid residue, the third structural domain further comprises a third mutation introduced by a substitution of a third amino acid residue with a hydrophilic amino acid residue or a hydrophobic amino acid residue.

More preferably, the third amino acid residue is a cysteine residue.

Preferably, the third mutation comprising any one of the followings: a substitution of the cysteine residue at position 125 with a serine residue (Ser, S); a substitution of the cysteine residue at position 125 with an alanine residue (Ala, A); a substitution of the cysteine residue at position 125 with a leucine residue (Leu, L).

Preferably, in C-terminus downstream area of the first amino acid residue, the third structural domain further comprises a third mutation introduced by a substitution of a third amino acid residue with a hydrophilic amino acid residue or a hydrophobic amino acid residue.

Preferably, the third amino acid residue is a cysteine residue, and the third mutation comprising any one of the followings: a substitution of the cysteine residue with a serine residue (Ser, S); a substitution of the cysteine residue with an alanine residue (Ala, A); a substitution of the cysteine residue with a leucine residue (Leu, L).

More preferably, the first structural domain comprises an amino acid sequence identical to any one of amino acid sequences of SEQ ID NOs: 2 to 6; the second structural domain comprises an amino acid sequence identical to any one of amino acid sequences of SEQ ID NOs: 7 to 10; the third structural domain an amino acid sequence identical to any one of amino acid sequences of SEQ ID NOs: 11 to 35.

More preferably, comprising an amino acid sequence with at least 80%, 85%, 90%, 95% or 99% similarity to any one of amino acid sequences of SEQ ID NOs: 36 to 57.

Another aspect of the present invention provides a medicine composition, comprising any one of the aforementioned polypeptides and a pharmaceutically acceptable carrier.

Yet another aspect of the present invention provides a use of the aforementioned medicine composition, wherein the medicine composition used for preparing a medical or cell therapeutic composition to treat, prevent or relieve a disease or a disorder caused by diminished immune cell activity.

Preferably, the immune cell comprises natural killer cells, NK-T cells, T cells, B cells, macrophages, dendritic cells, oligodendrocyte, cytokine induced killer cells, or γδT cells.

Preferably, the disease comprises a proliferative disorder including hematologic malignancy, solid tumors or metastatic tumors.

In the present invention, with introduction of a disulfide bond bridging the interleukin-2 receptor subunit α-binding domain and the non-interleukin-2 receptor subunit α-binding domain of IL-2, a polypeptide being more tended to activate IL-2 receptor subunits β and γ is created. Such polypeptide is not only able to activate effector T cells by stimulating proliferation thereof, but also improve thermal stability, solubility and productivity of the polypeptide itself. By means of this, times and dosage of injection during a cancer therapy are both reduced, and side effects are also ameliorated when achieving the expected therapeutic effects. The polypeptide can also be applied to cell therapy. When the polypeptide is supplemented to culture media of immune cells, cell viability of T cells is significantly enhanced. With less times and amounts of the supplemented polypeptide to culture media, immune cell preparation will be less time-consuming and low-cost.

### BRIEF DESCRIPTION OF THE DRAWINGS

FIG. 1A is a protein structure diagram to demonstrate a 3D structure of human interleukin-2 binding to IL-2 receptor;

FIG. 1B is a protein structure diagram to demonstrate a 3D structure of a mutant human interleukin-2 binding to IL-2 receptor;

FIG. 2 is a protein structure diagram to demonstrate a 3D structure of a mutant human interleukin-2;

FIG. 3 is a protein structure diagram to exemplarily demonstrate a relative position of the introduced disulfide bond in a mutant human interleukin-2;

FIG. 4 is a differential scanning calorimetry plot for comparison of melting temperatures of comparative example 1, 4 and example 3; and

FIG. 5 is a bar chart to parallelly compare cytotoxicity of immune cells treated with Example 2 and comparative example 4 against tumor cells.

### DETAILED DESCRIPTION OF THE INVENTION

### 1. Definitions

The term “peptide”, “polypeptide” and “protein” used herein can be switched, and refers to any polymeric pattern of amino acids in any length, and comprises coding and non-coding amino acids, chemically or biochemically-modified or -derived amino acids, or a polypeptide having a main chain with any type of modification.

The term “interleukin-2 receptor subunit α-binding domain”, unless otherwise defined, refers to a sequence segment of human interleukin-2 protein, as SEQ ID NO: 1, that is capable of binding to IL-2 receptor to activate downstream signaling pathway; to elaborate, an amino acid sequence corresponding thereto in a human interleukin-2 is protein sequence ³⁵KLTRMLTFKFYMPKKATELKHLQCLEEELKPLEEVLNL.

The term “non-interleukin-2 receptor subunit α-binding domain”, unless otherwise defined, refers to a sequence segment of human interleukin-2 protein, as SEQ ID NO: 1, that is not capable of binding to IL-2 receptor; to elaborate, an amino acid sequence corresponding thereto in a human interleukin-2 protein sequence is ¹APTSSSTKKTQLQLEHLLLDLQMILNGINNYKNP or ⁷³AQSKNFHLRPRDLISNINVIVLELKGSETTFMCEYADETATIVEFLNRW ITFCQSIISTLT.

Sequence similarity percentage between one polypeptide and another one polypeptide used herein, when comparing two sequences, refers that percentages of amino acids are identical, and these amino acids are at same positions; there are a variety of approaches to measure similarity between different sequences, including websites such as ncbi.nlm.nili.gov/BLAST, ebi.ac.uk/, Tools/msa/tcoffee/, ebi.ac.uk/Tools/msa/muscle/, mafft.cbrc.jp/alignment/software/, or any other methods or software available on the world wide web.

The term “hydrophilic amino acid” refers to amino acid containing a side chain of hydropathy index lower than 0, wherein the hydropathy index is measured according to ΔₜᵣGm^{θ} of phase transition of steam to water, and internal distribution of amino acids when a protein is folded; examples of “hydrophilic amino acid” in line with the above definition includes: serine (Ser, S; −0.8), threonine (Thr, T; −0.7), tyrosine (Tyr, Y; −1.3), asparagine (Asn, N; −3.5), glutamine (Gln, Q; −3.5), histidine (His, H; −3.2), tryptophan (Trp, W; −0.9) or proline (Pro, P; −1.6).

The term “hydrophobic amino acid” refers to amino acid containing a side chain of hydropathy index higher than 0, and examples of “hydrophobic amino acid” in line with the above definition includes: alanine (Ala, A; 1.8), valine (Val, V; 4.2), leucine (Leu, L; 3.8), Isoleucine (Ile, I; 4.5), methionine (Met, M; 1.9) or phenylalanine (Phe, F; 2.8).

The term “heterogenous polypeptide” used herein, unless otherwise defined, refers to an amino acid sequence unfound within a natural protein sequence.

The term “purification tag” used herein, unless otherwise defined, refers to an amino acid sequence linked to a recombinant protein by using genetic recombination technique; according to uses thereof, the “purification tag” can be categorized as followings: “affinity tag” refers to a protein tag capable of altering a recombinant protein's affinity to a specific binding site, thereby enabling use of affinity techniques to purify the recombinant protein, and includes chitin binding protein (CBP), maltose binding protein (MBP), Strep tag, glutathione-S-transferase (GST), or polyhistidine (ploy (His)); “solubilization tag” refers to a protein tag capable of facilitating correct protein folding and preventing formation of inclusion body, and includes thioredoxin (TRX) or poly (NANP); “chromatography tag” refers to a protein tag capable of altering a recombinant protein's chromatographic characteristics, thereby providing differential resolution in a specific isolation technique, and includes FLAG tag or His-tag; “epitope tag” refers to a protein tag capable of altering a recombinant protein's immune response, thereby enabling production of high-affinity antibodies among various species, and includes ALFA tag, V5 tag, Myc tag, HA tag, dot tag, T7 tag or NE tag; “fluorescence tag” refers to a protein tag capable of providing a recombinant protein with visual reading under specific optical condition, and includes GFP, RFP or YFP.

The term “recombination” used herein, unless otherwise defined, refers to a product of a specific nucleic acid (DNA or RNA) construct by any combination of steps including cloning, restriction, polymerase chain reaction (PCR) and/or ligation, and the construct contains a sequence distinguishing from endogenous coding or non-coding sequence discovered in natural system. A DNA sequence encoding an amino acid sequence can be constructed from a cDNA fragment or a series of synthetic oligonucleotides so as to express the synthetic nucleic acid in a recombinant transcription unit in a cellular or a cell-free translation/transcription system.

The terms “treatment”, “treating” used herein indicate to achieve pharmaceutical and/or physiological effects in anticipation, and particularly for completely or almost completely curing undesirable effects of a disease or resulting from the disease.

The term “prevention” used herein, unless otherwise defined, indicates to completely or almost completely stop disease or disorder from occurring. For example, when a subject has not been diagnosed of disease or disorder, or is suspected to have disease or disorder but not resulting in the disease or disorder, a preventive intervention is provided to prevent the disease or disorder from occurring.

The term “relieve” indicates to suppress or ameliorate development of disease or disorder when a subject has not been diagnosed of the disease or disorder, or is suspected to have the disease or disorder but not resulting in the disease or disorder; namely, progression of the disease or disorder is slowed down, or recession of the disease or disorder is induced.

The term “individual” and “subject” used herein are switchable, and refer to a mammalian entity in anticipation of being diagnosed, curing or conducting therapy; the mammalian entity comprises human, non-human primate, rodent (e.g. rat or mouse), lagomorph (e.g. rabbit), ungulate (e.g. cow, sheep, swine, horse, goat and so on).

Before further elaborating on the present invention, it should be understood that the present invention is not limited to the specific embodiments described herein and is therefore subject to variation. It should also be noted that the terminology used in this document is solely for the purpose of illustrating specific embodiments and is not intended to be restrictive, as the scope of the invention will be limited only by the appended claims.

When a range of values is provided, it should be understood that the present invention encompasses every intermediate value between the upper and lower limits of the range (unless explicitly indicated otherwise in the context, precise to one-tenth of the unit of the lower limit), as well as any other stated or intermediate values within the range. The upper and lower limits of such smaller ranges may independently be included within the smaller ranges and are also encompassed by the present invention, subject to any explicit exclusions stated within the range. If the stated range includes one or both of the boundary limits, the invention also encompasses ranges that exclude one or both of those boundary limits.

Unless otherwise defined, all technical and scientific terms used herein have the same meaning as commonly understood by those skilled in the art to which this invention pertains. While methods and materials similar or equivalent to those described herein can be used in the practice or testing of the present invention, the preferred methods and materials are now described.

Of note, unless otherwise provided, singular article “a”, “an” or “the” used herein and in the appended claims comprises a plurality of the indicated objects; therefore, when referring to “a interleukin-2 receptor subunit α-binding domain”, one or plural sequences of such polypeptides are included, and one or plural types of interleukin-2 receptor subunit α-binding domains and equivalents thereof that is acknowledged by those skilled in the art; it should be further noted, claims of the patent application can be designed to exclude any optional elements; accordingly, this statement is intended to serve as a foundational basis for combining elements of the claims with statements using exclusory terms such as “only,” “solely,” or similar expressions, or by employing “negative” limitations.

It should be understood that, for clarity, certain features of the present invention described in the context of individual embodiments may also be provided in combination within a single embodiment. Conversely, various features of the present invention described in the context of a single embodiment may likewise be provided individually or in any suitable combination for convenience. All combinations belonging to the embodiments of the present invention are specifically included and disclosed herein, as if each and every combination were individually and explicitly disclosed. Additionally, all sub-combinations of the various embodiments and their elements are also explicitly included and disclosed herein, as if each and every sub-combination were individually and explicitly disclosed.

### 2. Polypeptide

The first aspect of the present invention is related to a polypeptide, comprising:
(1) a first structural domain, comprising an amino acid sequence with at least 80%, 85%, 90%, 95% or 99% similarity to a first amino acid sequence at position 1 to position 34 of SEQ ID NO: 1, wherein the first amino acid sequence represents one of non-interleukin-2 receptor subunit α-binding domains (non-IL-2Rα binding domain hereinafter);
(2) a second structural domain, configured at C-terminus of the first structural domain, comprising an amino acid sequence with at least 80%, 85%, 90%, 95% or 99% similarity to a second amino acid sequence at position 35 to position 72 of SEQ ID NO: 1, wherein the second amino acid sequence represents an interleukin-2 receptor subunit α-binding domain (IL-2Rα binding domain hereinafter); and
(3) a third structural domain, configured at C-terminus of the second structural domain, comprising an amino acid sequence with at least 80%, 85%, 90%, 95% or 99% similarity to a third amino acid sequence at position 73 to position 133 of SEQ ID NO: 1, wherein the third amino acid sequence represents one of non-interleukin-2 receptor subunit α-binding domains, and a disulfide bond is introduced to bridge the third structural domain and the first structural domain or the second structural domain, or the disulfide bond is introduced into the third structural domain.

In various embodiments, the third structural domain is introduced of a first mutation by a substitution of a first amino acid residue with a cysteine residue, wherein the polypeptide is further introduced of a second mutation by a substitution of a second amino acid residue with another cysteine residue, and the second mutation optionally happens in the first structural domain, the second structural domain or the third structural domain, thereby forming a disulfide bond between the cysteine residue introduced by the first mutation and the cysteine residue introduced by the second mutation.

Under a normal circumstance, IL-2 receptor is a heteromer composed of three distinguishable subunits α, β, γ. Since IL-2 demonstrates high affinity to IL-2 receptor subunit α (IL-2Rα hereinafter), the cell signaling remains functional when cytokine is completely reduced, and a IL-2 reservoir forms on the cell surface to facilitate IL-2 recovering thereto. However, as T cells compete for a finite amount of cytokines, continuous activation of IL-2Rα eventually transform T cells into CD8⁺ T cells. Persistent number expansion of regulatory T cells would consume T cells and such a process suppresses activity of effector T cells.

In one or various embodiments of the followings, the disulfide bond introduced by the aforesaid mutations alters 3D structure of the IL-2Rα binding domain and the non-IL-2Rα binding domain, whose structure is distinguishable from 3D structure of a wild-type IL-2, and alters binding affinity of IL-2Rα binding domain to IL-2Rα when the polypeptide binds to IL-2 receptor on cell surface of a T cell.

In preferred embodiments, to improve structural stability of the polypeptide, and to alter binding affinity of IL-2Rα binding domain to IL-2Rα, suitably, the first amino acid residue comprises a asparagine residue (Asn, N) at position 77, an aspartate residue (Asp, D) at position 84, a isoleucine residue (Ile, I) at position 92, Lysine residue (Lys, K) at position 97, a threonine residue (Thr, T) at position 111, a phenylalanine residue (Phe, F) at position 117 or a leucine residue (Leu, L) at position 132; the second amino acid residue comprises a glutamine residue (Gln, Q) at position 11, a leucine residue (Leu, L) at position 17, a leucine residue (Leu, L) at position 18, a tyrosine residue (Tyr, Y) at position 31, a lysine residue (Lys, K) at position 43, a phenylalanine residue (Phe, F) at position 44, a leucine residue (Leu, L) at position 56 or a leucine residue (Leu, L) at position 80.

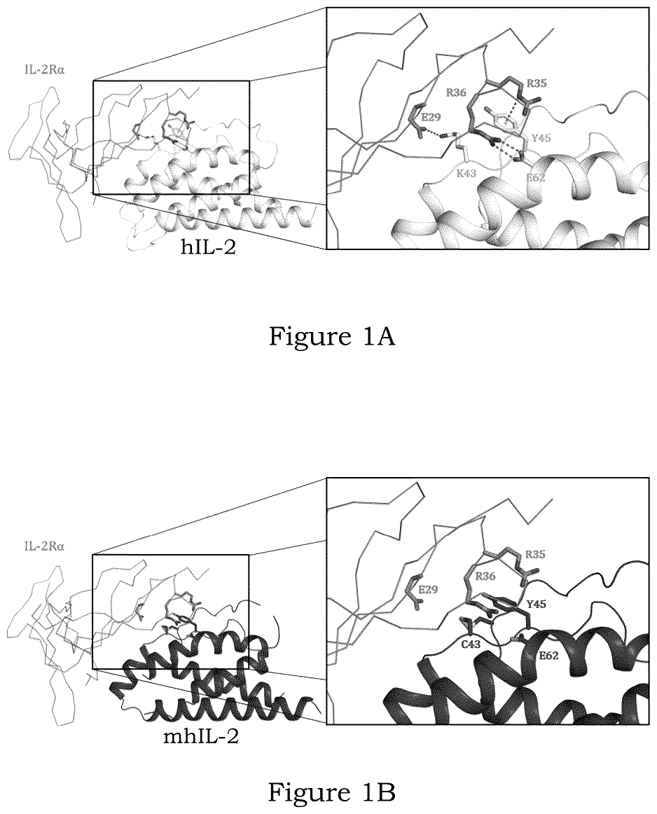

To further elaborate, determination of the second amino acid residue depends on that the residue's side chain is capable of forming a non-covalent bond with IL-2 binding site on the IL-2Rα, or that there is another amino acid residue with decisive effects on IL-2 binding site's structure in proximity of the second amino acid residue. With reference to FIG. 1A, taking wild-type human interleukin-2 (hIL-2 hereinafter) for instance, those amino acid residues on protein sequence of hIL-2, such as lysine residue at position 43, tyrosine residue at position 45 or glutamate residue at position 62, are capable of forming non-covalent bond with the corresponding amino acid residues on amino acid sequence of IL-2Rα such as glutamate at position 29 and arginine at position 35 or 36, respectively, are revealed in protein structure by X-ray crystallography.

As shown in FIGS. 1A and 1B, after introduction of the first mutation and the second mutation, as exemplified by cysteine residues at positions 43 and 111, a stable structure is formed and hydrogen bond between lysine at position 43 of the IL-2Rα binding domain and glutamate residue at position 29 of IL-2Rα is disrupted. Accordingly, the polypeptide shows more tendency to bind to IL-2Rβ and IL-2Rγ.

In the first aspect of the present invention, the sequence arrangements as described hereinabove are intended to alter binding affinity to IL-2Rα and/or other biological functions of the IL-2Rα binding domain.

In certain embodiments, such sequence arrangements are intended to create IL-2 mutants having higher binding affinity to IL-2Rα and/or IL-2Rβγ. Such IL-2 mutants can be created by introduction of appropriate mutations (e.g. deletion, insertion or substitution) into nucleic acid encoding IL-2, or by peptide synthesis technique. In view of the characteristics on improvement, the IL-2Rα binding domain and/or the non-IL-2Rα binding domain are further subject to amino acid mutations other than the first mutation and the second mutation, and subject to screening based on maintenance or improvement of its binding affinity, or its 3D structural stability.

Conceivably, sequence of the polypeptide is not limited to introduction of mutant amino acid in a full-length IL-2 polypeptide, and the sequence of the polypeptide can be any one of the following sequence arrangements:
(a) C-terminus of the first structural domain links to N-terminus of the second structural domain, and C-terminus of the second structural domain links to N-terminus of the third structural domain;
(b) a heterogenous polypeptide is arranged at N-terminus of the first structural domain or at C-terminus of the third structural domain;
(c) the first structural domain further links to the second structural domain via a first linker, and the second structural domain further links to the third structural domain via a second linker;
(d) C terminus of the first structural domain or N-terminus of the third structural domain further links to the heterogenous polypeptide via a third linker.

The term “linker” used herein, unless otherwise defined, refers to oligopeptide or other means that are selectively linked to two polypeptide molecules, and a single-functioned polypeptide or a double-functioned polypeptide in a continuous polypeptide molecular form is created thereby. Some strategies include linking these polypeptide molecules in covalent bonding manner, but not limited to a linkage between N- and C-termini of proteins or protein structural domains through a polypeptide bond, a disulfide bond or a chemical cross-linker. Selection of an appropriate linker between two polypeptides upon specific condition depends on a variety of parameters, such as two polypeptides' properties including distance between N- and C-termini, and/or linker's effect on protein hydrolysis or oxidation, but not limited to this. Additionally, the linker also comprises amino acid residues providing flexibility.

The aforesaid linker can refer to the first linker, the second linker and/or the third linker, and they can be identical or distinguishable. The length of linker shall be sufficient to allow two polypeptide molecules to fold in correct structures so as to maintain activities thereof. In practice, the linker can be 2 to 30 amino acids long. The amino acid residues in a linker should not interfere with molecular activity of the polypeptide. Accordingly, the linker should possess a charge that is more consistent with the activity of the polypeptide molecule to avoid interfering with the folding pattern of the polypeptide molecule, or, to avoid bond formation or other interaction between the linker and one or more amino acid residues in the polypeptide molecule.

Feasibly, the linker comprises glycine-serine linker (a.k.a GS linker), which refers to a polymer of serial connected glycine and serine, such as (Gly-Ser)n, (GSGGS)n, (GGGGS)n or (GGGS)n, and n is an integer of at least 1. In other cases, the linker refers to glycine-alanine polymer, alanine-serine polymer or other flexible linkers.

In some embodiments, in C-terminus downstream area of the first amino acid residue, the third structural domain further comprises a third mutation introduced by a substitution of a third amino acid residue with a hydrophilic amino acid residue or a hydrophobic amino acid residue, wherein the third amino acid residue is a cysteine residue. Accordingly, dissociation property of the polypeptide is improved, solubility and melting temperature (Tm) are both enhanced, and the polypeptide are less tended to form inclusion bodies. Based on these property improvements, productivity of the polypeptide is gained due to enhancement of isolation and purification efficiency.

In certain embodiments, the hydrophilic amino acid residue is exemplified by serine residue (Ser, S), Threonine residue (Thr, T), tyrosine residue (Tyr, Y), Aspartate residue (Asn, N), glutamine residue (Gln, Q), histidine (His, H), tryptophan (Trp, W) or methionine (Met, M); the hydrophobic amino acid residue is exemplified by alanine residue (Ala, A), valine residue (Val, V), leucine residue (Leu, L), Isoleucine residue (Ile, I), proline residue (Pro, P) or phenylalanine residue (Phe, F); feasibly, the third mutation comprising a substitution of the cysteine residue at position 125 with a serine residue (Ser, S), an alanine residue (Ala, A), or a leucine residue (Leu, L).

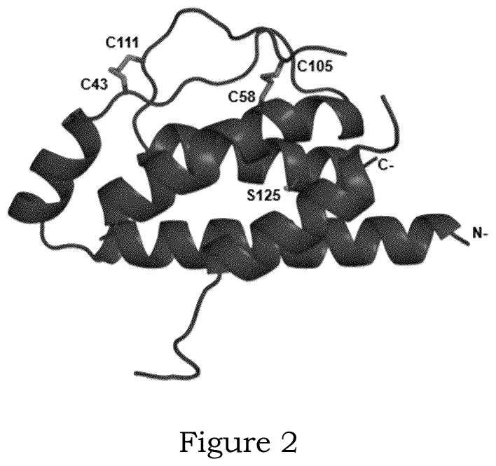

As shown in FIG. 2, in one example of the third mutation having substitution of the cysteine residue at position 125 with a serine residue, there are at least two disulfide bonds in the full-length protein sequence of a human IL-2 mutant (mhIL-2 hereinafter): cysteine residue at position 43-cysteine residue at position 111 (C43-C111), cysteine residue at position 58-cysteine residue at position 105 (C58-C105). As the cysteine residue at position 125 is substituted of a serine residue (C125S), hydrophilicity of C-terminal segment of mhIL-2 is increased, thereby enhancing solubility of mhIL-2.

In some preferred embodiments, the first structural domain comprises an amino acid sequence identical to any one of amino acid sequences of SEQ ID NOs: 2 to 6; the second structural domain comprises an amino acid sequence identical to any one of amino acid sequences of SEQ ID NOs: 7 to 10; the third structural domain an amino acid sequence identical to any one of amino acid sequences of SEQ ID NOs: 11 to 35.

In particular embodiments, when the first structural domain comprises an amino acid sequence identical to an amino acid sequence of SEQ ID NO: 2, the second structural domain comprises an amino acid sequence identical to any one of amino acid sequences of SEQ ID NOs: 8 to 10, and the third structural domain comprises an amino acid sequence identical to any one of amino acid sequences of SEQ ID NOs: 24 to 32; when the first structural domain comprises an amino acid sequence identical to any one of amino acid sequences of SEQ ID NOs: 3 to 6, the second structural domain comprises an amino acid sequence identical to an amino acid sequences of SEQ ID NO: 7, and the third structural domain comprises an amino acid sequence identical to any one of amino acid sequences of SEQ ID NOs: 11, 15 to 17, 21 to 23 and 33 to 35; when the first structural domain comprises an amino acid sequence identical to an amino acid sequence of SEQ ID NO: 2, the second structural domain comprises an amino acid sequence identical to any one of amino acid sequences of SEQ ID NO: 6, and the third structural domain comprises an amino acid sequence identical to any one of amino acid sequences of SEQ ID NOs: 18 to 20.

Taking a full-length mhIL-2 protein sequence as a practical solution, the polypeptide comprises an amino acid sequence with at least 80%, 85%, 90%, 95% or 99% similarity to any one of amino acid sequences of SEQ ID NOs: 36 to 57; preferably, the polypeptide comprises an amino acid sequence identical to any one of amino acid sequences of SEQ ID NOs: 36 to 57; more preferably, the polypeptide comprises an amino acid sequence identical to any one of amino acid sequences of SEQ ID NOs: 46 to 48.

Optionally, the polypeptide may be a fusion protein capable of binding to multiple types of interleukin receptors on a cell surface; preferably, the heterogenous polypeptide may be substituted of an interleukin family member, such as interleukin-1, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interleukin-18, interleukin-19, interleukin-20, interleukin-21, interleukin-22, interleukin-23, interleukin-24, interleukin-25, interleukin-26, interleukin-27, interleukin-28, interleukin-29, interleukin-30, interleukin-31, interleukin-32 or interleukin-33.

Additionally, the heterogenous polypeptide is substituted of a PEG or a serum half-life extension polypeptide so that the polypeptide's half-life is lengthened in circulation system of an individual, which is expected to reduce times of the polypeptide administration in the subsequent embodiments. Concretely, the serum half-life extension polypeptide may be a fragment crystallizable region (Fc region), serum albumin, albumin-binding protein or peptide, IgG, XTEN polypeptide, proline/alanine/serine-rich polypeptide (PAS), elastin-like polypeptide or PEGylation, but not limited to this.

Yet in addition, the heterogenous polypeptide is substituted of a purification tag, thereby improving solubility of the polypeptide in the protein expression systems and maintaining activities thereof during purification/isolation process, which facilitates enrichment and purification of the polypeptide in protein expression systems of the subsequent embodiments. Typically, the purification tag can be exemplified by His-tag, GST-tag, maltose-binding protein tag (MBP-tag), NusA-tag, or small ubiquitin-like modifier tag (SUMO-tag), but not limited to this.

### 3. Medicine Composition

Provided in the second aspect of the present invention is a medicine composition, comprising the polypeptide provided in the first aspect of the present invention and a pharmaceutically acceptable carrier. The term “pharmaceutically acceptable carrier” used herein refers to, in addition to active ingredients, the ingredients being non-toxic to the subject, and comprises buffer agent(s), vehicle(s), stabilizer(s) or antiseptic(s), but not limited to this.

In one or various embodiments, the medicine composition is administrated in an oral dosage form, an injective dosage form, an inhalation dosage form, or a regional/percutaneous dosage form.

### 4. Use of Medicine Composition

Provided in the third aspect of the present invention is a use of a medicine composition, wherein the medicine composition is the medicine composition provided in the second aspect of the present invention, and being used for preparing a medical or cell therapeutic composition to treat, prevent or relieve a disease or a disorder caused by diminished immune cell activity.

In various embodiments, the immune cell comprises natural killer cells, NK-T cells, T cells, B cells, macrophages, dendritic cells or oligodendrocyte,

The term “T cells” used herein refers to CD3⁺ immune cells including helper T cells (CD4⁺ cells), cytotoxic T cells (CD8⁺ cells), effector Th cells, memory Th cells, regulatory T cells (Treg) and natural killer T cells (NK-T cells).

Particularly, the immune cells may refer to effector Th cells, memory Th cells, regulatory T cells (Treg) or natural killer T cells (NK-T cells); optionally, the immune cells comprise cytokine induced killer cells (CIK) composed of NK cells, NK-T cells and T cells, or γδT cells.

Preferably, the disease comprises a proliferative disorder including hematologic malignancy, solid tumors or metastatic tumors. In concrete terms, the solid tumor may be a renal solid tumor selected from the group consisting of ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms' tumor), papillary adenoma, papillary renal cell carcinoma, renal oncocytoma, renal cell carcinoma, succinate dehydrogenase-deficient renal cell carcinoma and collecting duct carcinoma, or a skin solid tumor selected from the group consisting of cutaneous melanoma and mucosal melanoma, or a hematologic malignancy selected from the group consisting of leukemia, lymphoma, and multiple myeloma.

### 5. Nucleic Acid and Expression System

A nucleic acid encoding the polypeptide in the first aspect of the present invention is also provided herein, comprising RNA, DNA or cDNA, but not limited to this. The nucleic acid may be a vector, or inserted into a specific vector, and the vector type is not limited to plasmid, cosmid or YAC. In certain embodiments, the vector is applied to offer an expression vector for the polypeptide provided in the first aspect of the present invention in a host cell, a host organism and/or an expression system. Typically, the expression vector comprises the nucleic acid and one or more regulatory elements linked thereto, such as promoter, enhancer, or stop codon. The selection of elements and their sequences for expression in a specific host does not deviate from the common understanding of those skilled in the art. The regulatory elements and other elements that are functional or indispensable for expression of the polypeptide may be exemplified by promoter, enhancer, stop codon, integrative/conjugative element, selection marker, guide sequence or reporter gene.

The aforementioned nucleic acids may be prepared, according to amino acid sequence information of the polypeptide, or at least according to any one or a reasonable combination of amino acids sequences of SEQ ID NOs: 1 to 52, by well-known techniques (e.g. automated DNA synthesis and/or DNA recombination), and/or by isolation from suitable biological materials.

In certain embodiments, a host cell for expression of the polypeptide in the first aspect of the present invention is provided herein; of course, the host cell may contain the aforesaid nucleic acids or the aforesaid expression vectors; feasibly, the host cell may be a bacterial cell, a fungal cell, a yeast cell, or a mammalian cell.

Preferably, the bacterial cell may include Gram-negative bacteria such as E. coli., Proteus, or Pseudomonas, or Gram-positive bacteria such as Bacillus, Streptomyces, Staphylococcus, or Lactococcus.

Preferably, the fungal cell may include a cell from any one species under Trichoderma, Neurospora, Aspergillus; or a cell of any species under Saccharomyces such as, Saccharomyces Cerevisiae, Schizosaccharomyces, or Schizosaccharomyces pombe; or a cell of any species under Pichia, such as Pichia pastoris or Pichia methanolica; or a cell of any species under Hansenula.

Preferably, the mammalian cell may be selected from immortalized cell lines that is commonly used for protein expression and purification, such as HEK293 cell, CHO cell, BHK cell, HeLa cell, COS cell. In addition to the aforesaid cell lines, any other cells derived from amphibian cells, insect cells or plant cells that can be applied for expression of heterogenous proteins in the art may also be included.

The polypeptide provided in the first aspect of the present invention may be expressed in the aforesaid host cells, and then be isolated from the host cells before subject to any purification process. Alternatively, the polypeptide may be produced outside the cell. For example, the polypeptide may be produced in culture media of the host cell, or in a cell-free protein synthesis system, and then isolated from the host cells before subject to any purification process.

Methods for transformation or transfection of the expression vector, purification tag, methods for induced expression and culture conditions are well-acknowledged in the art. Similarly, suitable techniques required to produce the polypeptide, including isolation and purification, are also well-known for those skilled in the art. However, on top of using the host cell or cell-free expression system, the polypeptide may be obtained using a solid-phase or a liquid-phase chemical synthesis method, or other well-known protein production processes in the art.

The following examples are described hereinafter sole to illustrate the present invention, but are not intended to limit implementation thereof.

### 1. Introduction of First Mutation and Second Mutation (Introduction of Disulfide Bond)

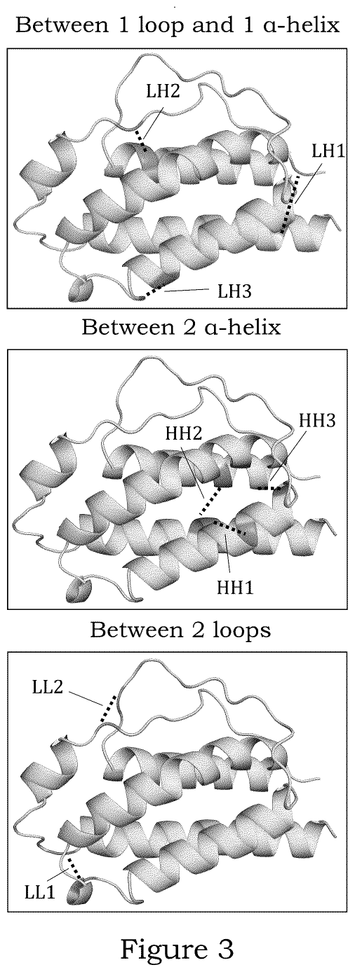

In the following examples, human interleukin-2 (hIL-2 hereinafter) was used as a template protein to introduce a mutant amino acid residue so as to create the polypeptide with an additional disulfide bond. FIG. 3 illustrates that there are 4 α-helixes in hIL-2, and one loop formed between each two neighboring α-helixes. The additional disulfide bond was introduced with first mutation and second mutation in the template protein's structure.

FIG. 3 further indicates that suitable positions for disulfide bond design are located between one loop and one α-helix, two α-helixes, or two loops, and positions of disulfide bonds can be found in Table 1.

TABLE 1

| | Positions of disulfide bond |
|---|---|
| | (in hIL-2) |
| IL-2-LH1 | Q11^{Helix}/L132^{Loop} |
| IL-2-HH1 | L17^{Helix}/I95^{Helix} |
| IL-2-HH2 | L18^{Helix}/C125^{Helix} |
| IL-2-LL1 | Y31^{Loop}/N77^{Loop} |
| IL-2-LL2 | K43^{Loop}/T111^{Loop} |
| IL-2-LH2 | F44^{Loop}/F117^{Helix} |
| IL-2-HH3 | L56^{Helix}/K97^{Helix} |
| IL-2-LH3 | L80^{Loop}/D84^{Helix} |

Shown in Table 2 are amino acid sequences of hIL-2 with introduction of the additional disulfide bond, and they are created by substitution of amino acid at the position of the expected disulfide bond with the amino acid sequence hIL-2 as the reference sequence. In Table 1, the underlined alphabets represent mutant residues corresponding to disulfide bond listed in Table 1.

### 2. Introduction of Third Mutation

Hereinafter, the IL-2-LH2, IL-2-HH1, IL-2-HH3, IL-2-LH3, IL-2-LL1, IL-2-LL2 were further introduced of a third mutation at position 125 so as to improve solubility of the polypeptide furthermore (Experimental examples 1 to 8). On the other hand, besides taking wild-type hIL-2 as comparative example 1, the wild-type hIL-2 was also further introduced of a third mutation at position 125 by substitution with different amino acid residues (S, A, L), respectively, to serve as comparative examples 2 to 4.

Listed in Table 3 are amino acid sequences of wild-type hIL-2, hIL-2 with the third mutation, and mhIL-2 with the additional disulfide bond. The underlined alphabets represent mutant residues of the first mutation, the second mutation and the third mutation.

TABLE 3

| | Sequences |
|---|---|
| Comparative | APTSS-STKKT-QLQLE-HLLLD-LQMIL-NGINN-YKNPK-LTRML-TFKFY-MPKKA |
| example 1 | TELKH-LQCLE-EELKP-LEEVL-NLAQS-KNFHL-RPRDL-ISNIN-VIVLE-LKGSE |
| | TTFMC-EYADE-TATIV-EFLNR-WITFC-QSIIS-TLT |
| Comparative | APTSS-STKKT-QLQLE-HLLLD-LQMIL-NGINN-YKNPK-LTRML-TFKFY-MPKKA |
| example 2 | TELKH-LQCLE-EELKP-LEEVL-NLAQS-KNFHL-RPRDL-ISNIN-VIVLE-LKGSE |
| | TTFMC-EYADE-TATIV-EFLNR-WITFS-QSIIS-TLT |
| Comparative | APTSS-STKKT-QLQLE-HLLLD-LQMIL-NGINN-YKNPK-LTRML-TFKFY-MPKKA |
| example 3 | TELKH-LQCLE-EELKP-LEEVL-NLAQS-KNFHL-RPRDL-ISNIN-VIVLE-LKGSE |
| | TTFMC-EYADE-TATIV-EFLNR-WITFA-QSIIS-TLT |
| Comparative | APTSS-STKKT-QLQLE-HLLLD-LQMIL-NGINN-YKNPK-LTRML-TFKFY-MPKKA |
| example 4 | TELKH-LQCLE-EELKP-LEEVL-NLAQS-KNFHL-RPRDL-ISNIN-VIVLE-LKGSE |
| | TTFMC-EYADE-TATIV-EFLNR-WITFL-QSIIS-TLT |
| Experimental | APTSS-STKKT-QLQLE-HLLLD-LQMIL-NGINN-YKNPK-LTRML-TFCFY-MPKKA |
| example 1 | TELKH-LQCLE-EELKP-LEEVL-NLAQS-KNFHL-RPRDL-ISNIN-VIVLE-LKGSE |
| | TTFMC-EYADE-CATIV-EFLNR-WITFS-QSIIS-TLT |
| Experimental | APTSS-STKKT-QLQLE-HLLLD-LQMIL-NGINN-YKNPK-LTRML-TFCFY-MPKKA |
| example 2 | TELKH-LQCLE-EELKP-LEEVL-NLAQS-KNFHL-RPRDL-ISNIN-VIVLE-LKGSE |
| | TTFMC-EYADE-CATIV-EFLNR-WITFA-QSIIS-TLT |
| Experimental | APTSS-STKKT-QLQLE-HLLLD-LQMIL-NGINN-YKNPK-LTRML-TFCFY-MPKKA |
| example 3 | TELKH-LQCLE-EELKP-LEEVL-NLAQS-KNFHL-RPRDL-ISNIN-VIVLE-LKGSE |
| | TTFMC-EYADE-CATIV-EFLNR-WITFL-QSIIS-TLT |
| Experimental | APTSS-STKKT-QLQLE-HLLLD-LQMIL-NGINN-YKNPK-LTRML-TFKCY-MPKKA |
| example 4 | TELKH-LQCLE-EELKP-LEEVL-NLAQS-KNFHL-RPRDL-ISNIN-VIVLE-LKGSE |
| | TTFMC-EYADE-TATIV-ECLNR-WITFL-QSIIS-TLT |
| Experimental | APTSS-STKKT-QLQLE-HCLLD-LQMIL-NGINN-YKNPK-LTRML-TFKFY-MPKKA |
| example 5 | TELKH-LQCLE-EELKP-LEEVL-NLAQS-KNFHL-RPRDL-ISNIN-VCVLE-LKGSE |
| | TTFMC-EYADE-TATIV-EFLNR-WITFL-QSIIS-TLT |
| Experimental | APTSS-STKKT-QLQLE-HLLLD-LQMIL-NGINN-YKNPK-LTRML-TFKFY-MPKKA |
| example 6 | TELKH-CQCLE-EELKP-LEEVL-NLAQS-KNFHL-RPRDL-ISNIN-VIVLE-LCGSE |
| | TTFMC-EYADE-TATIV-EFLNR-WITFL-QSIIS-TLT |
| Experimental | APTSS-STKKT-QLQLE-HLLLD-LQMIL-NGINN-YKNPK-LTRML-TFKFY-MPKKA |
| example 7 | TELKH-LQCLE-EELKP-LEEVL-NLAQS-KNFHC-RPRCL-ISNIN-VIVLE-LKGSE |
| | TTFMC-EYADE-TATIV-EFLNR-WITFL-QSIIS-TLT |
| Experimental | APTSS-STKKT-QLQLE-HLLLD-LQMIL-NGINN-CKNPK-LTRML-TFKFY-MPKKA |
| example 8 | TELKH-LQCLE-EELKP-LEEVL-NLAQS-KCFHL-RPRDL-ISNIN-VIVLE-LKGSE |
| | TTFMC-EYADE-TATIV-EFLNR-WITFL-QSIIS-TLT |

### 3. Protein Preparation

The mutant form of hIL-2 (mhIL-2) was expressed in Pichia pastoris. In brief, the mhIL-2 was prepared by using pPICZαA expression vector (Zeiocin-resistant) transformed in X-33 strain in YPD media.

First of all, the X-33 strain was cultured in 400 mL culture media at 30° C. for 48 hours, and then was transferred to 1100 mL culture media and cultured at 30° C. for another 60 hours. Subsequently, cell pellets were collected by 15-minute centrifugation at 3500 rpm, and was transferred to 1000 mL induction media BMM. Methanol was added to the BMM so as to induce protein expression at 20° C. for 72 hours, and supernatants of the BMM were collected after centrifugation.

The protein was purified by using salt-tolerable cation exchange column (Capto MMC) and RP-HPLC. In short, the supernatant was mixed with 10× binding buffer (50 mM sodium acetate, pH4.0) at a volume ratio of 10:1, and pH value was adjusted to the same as that of binding buffer. The mixture was then centrifuged at 4° C., 8000 rpm and passing through filter paper, and the filtered supernatant was preserved for subsequent loading into the cation exchange column balanced with binding buffer. Extracting buffer (50 mM ammonium bicarbonate in Buffer B) was applied to perform a gradient extraction of the target protein. The extracted target protein was resolved in 15% Glycine SDS-PAGE so as to ensure the identity thereof. Finally, a secondary purification of the target protein was performed using RP-HPLC with column C18. Gradient washing by using water (Buffer A) and acetonitrile (Buffer B) allowed production of the target protein at 95% purity. The purified target protein was then subject to freeze-dry so as to be stored at −80° C. in a powder form until use.

### 4. Protein Property Analysis

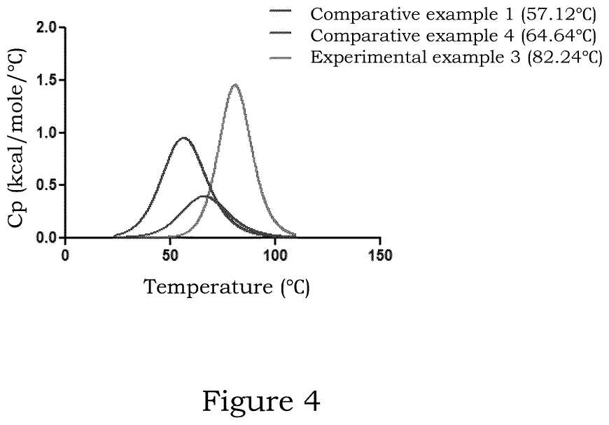

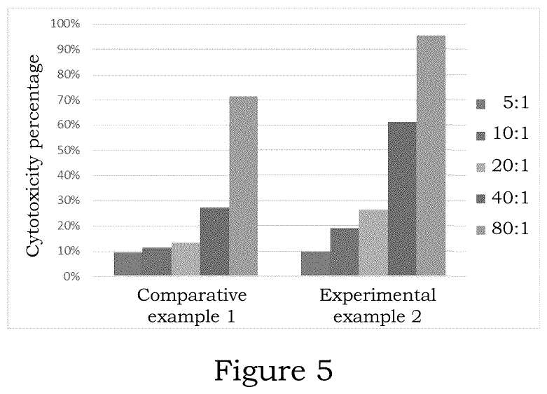

Protein melting temperature was analyzed by differential scanning calorimetry (DSC). As shown in Table 4 and FIG. 4, when compared with comparative example 1, the comparative example 4 (C125L hereinafter) having substitution of cysteine residue with leucine residue at position 125 demonstrated a significant melting temperature difference (ΔTm1) of +7.52, which indicates an enhancement of thermostability of mhIL-2. In contrast, substitution of cysteine residue with serine residue (C125S hereinafter) or alanine residue (C125A hereinafter) at position 125 rather undermined thermostability of mhIL-2, and led to a decrease of mhIL-2 melting temperature, wherein the ΔTm1 were −8.76 and −7.73, respectively.

Besides, when compared with comparative examples 2 to 4, the melting temperature differences (ΔTm2) of experimental examples 1 to 3 were significantly increased, and reached +12.25, +17.08 and +17.60, respectively. In particular, the ΔTm2 of experimental example 3 even achieved at +25.12 when compared with comparative example 1, which showed a prominent thermostability enhancement in contribution of the additional disulfide bond in IL-2-LL2.

Steps of the aforesaid protein solubility assay are illustrated as the following: obtaining 10 μL PBS (pH7.4) to dissolve mhIL-2 protein powder with already-known weight in an Eppendorf, and define a theoretical concentration value after the protein powder is thoroughly dissolved. The Eppendorf was then subject to high-speed centrifugation at 4° C., 13500 rpm for 10 minutes, and supernatant was transferred to another clean Eppendorf. A280 concentration of the supernatant was measured for twice, and an average value was calculated to be the protein concentration. The results of protein solubility assay can be found in Table 4. When compared with comparative example 1, solubilities of comparative examples 2 to 4 (C125S, C125A, C125L, respectively) were increased up to 1.6 to 3.0 folds. In another aspect, solubilities of experimental examples 1 to 3 with additional disulfide bonds were even increased up to 3.7 to 5.6 folds.

TABLE 4

| | | | | | | | | | CTLL-2 |
|---|---|---|---|---|---|---|---|---|---|
| | | Calculated | Expected | Mw | | Thermostability | | | Proliferation |
| | productivity | Mw | Mw | variation | Solubility | Tm | ΔTm1 | ΔTm2 | (×10⁶ |
| | (mg/L) | (Da) | (Da) | (Da) | (mg/mL) | (° C.) | (° C.) | (° C.) | U/mg) |
| comparative | 2.4 | 15416.01 | 15415.30 | −0.71 | 10.95 | 57.12 | — | — | 0.61 ± 0.19 |
| example 1 | | | | | | | | | |
| comparative | 3.5 | 15399.95 | 15399.09 | −0.86 | 26.25 | 48.36 | −8.76 | — | 0.26 ± 0.02 |
| example 2 | | | | | | | | | |
| comparative | 4.0 | 15383.95 | 15383.33 | −0.62 | 17.61 | 49.39 | −7.73 | — | 0.43 ± 0.08 |
| example 3 | | | | | | | | | |
| comparative | 3.6 | 15426.03 | 15425.19 | −0.84 | 32.86 | 64.64 | +7.52 | — | 0.75 ± 0.11 |
| example 4 | | | | | | | | | |
| experimental | 5.2 | 15374.94 | 15373.95 | −0.99 | 40.77 | 60.61 | +3.49 | +12.25 | 1.72 ± 0.37 |
| example 1 | | | | | | | | | |
| experimental | 8.8 | 15358.94 | 15357.95 | −0.99 | 40.65 | 66.47 | +9.35 | +17.08 | 1.80 ± 0.22 |
| example 2 | | | | | | | | | |
| experimental | 18.2 | 15401.03 | 15400.04 | −0.99 | 60.87 | 82.24 | +25.12 | +17.60 | 2.23 ± 0.55 |
| example 3 | | | | | | | | | |

### 5. Proliferation of Immune Cells

Specific activity assay was conducted using mouse cytotoxic T cell line CTLL-2 in order to evaluate the polypeptide's influence on cell proliferation of immune cells, and the steps are illustrated as the following:
(1) Comparative examples 1 to 4 were diluted by RPMI-1640 to serve as hIL-2 standard sample, and experimental examples 1 to 4 were diluted by RPMI-1640 to serve as mhIL-2 test samples. Serial dilutions of both standard sample and test samples started from 100 ng/mL, and sample volume of each culture well in a 96-well plate was 50 μL. Each tested concentration was prepared in 3 repeats;
(2) CTLL-2 cells at log phase were harvested for cell proliferation assay;
(3) Cell density was adjusted to be 8×10⁴ cells/mL, and 50 μL of cell suspension was pipetted into each culture well, thereby reaching a final volume of 100 μL. The CTLL-2 cells were cultured for 48 hours;
(4) Ten microliters of CCK-8 regent were added to each culture well. The 96-well plate was placed on a shaker for 1-minute shaking, and then subject to 4-hour culturing at 37° C., 5% CO₂. Subsequently, the 96-well plate was subject to measurement of absorbance at OD450-OD600;
(5) Results analysis: the dilution concentrations of the hIL-2 standard sample and the mhIL-2 test samples were plotted on the x-axis, and the OD value of the tested cells were plotted on the y-axis. The dilution concentration corresponding to 50% of the maximum OD value for the standard sample was determined so that the corresponding dilution concentration at 50% of the maximum OD value for the test sample was to be identified.

The test results can be found in Table 4. The specific activity of the comparative example 1 was 0.61±0.19×10⁶ U/mg. In contrast, the specific activities of comparative examples 2 and 3 were only 0.26±0.02×10⁶ U/mg and 0.43±0.08×10⁶ U/mg, respectively. Cell viability of CTLL-2 was rather decreased, which indicated that mutant forms including C125S and C125A not only undermined thermostability of mhIL-2, but also reduced cell proliferative activity thereof. The specific activity of comparative example 4 was 0.75±0.11×10⁶ U/mg, which indicated that the mutation C125L enhanced thermostability of mhIL-2, and also increased cell viability of CTLL-2 with significant raise of 22.9%.

Compared with comparative examples 1 to 4, experimental examples 1 to 3 demonstrated that the additional disulfide bond between IL-2Rα binding domain and non-IL-2Rα binding domain further increased cell proliferative activity of mhIL-2, and corresponding specific activities were 1.72±0.37×10⁶ U/mg, 1.80±0.22×10⁶ U/mg and 2.23±0.55×10⁶ U/mg, respectively. They promoted cell viability of CTLL-2 with significant raises of CTLL-2 by 181.9%, 195.1% and 265.6%, respectively.

### 6. Cytotoxicity Assay

The specific cytotoxicity was assayed based on peripheral blood mononuclear cells (PBMC) in order to evaluate the cytotoxic effects of the polypeptide-activated immune cells over HepG2 cells, and the steps are illustrated as the following:
(1) Ficoll was used to isolate 20 mL of peripheral venous blood. There were 4 layers from tube bottom to liquid surface after centrifugation, including, sequentially, red blood cell and granulocyte layer, stratified fluid layer, mononuclear cell layer and plasma layer;
(2) The mononuclear cell layer was extracted and adjusted to be at cell density of 2×10⁶ cells/mL. On that day, 1000 U/mL of IFN-γ was supplemented to culture media, and the mononuclear cells were cultured at 37° C., 5% CO₂ for 24 hours. Before the mononuclear cells were subject to another 48-hour cell culturing, 100 ng/ml of anti-CD3 monoclonal antibody, 300 IU/mL of mhIL-2 test sample (experimental example 2) and hIL-2 standard sample (comparative example 1) were supplemented to culture media;
(3) In the first week, 300 IU/mL of mhIL-2 test sample and hIL-2 standard sample were supplemented to culture media once every 4 days, and 1000 IU/mL of mhIL-2 test sample and hIL-2 standard sample were supplemented to culture media once every 4 days in the second to the third week;
(4) One day prior to co-culture of the mononuclear cells and HepG2 cells, the HepG2 cells expressing GFP were attached to 96-well plate, and each well was seeded of HepG2 cells at cell density of 8×10⁴ cells/mL in 100 μL;
(5) On the day of co-culture, the activated immune cells and the tumor cells (HepG2 cells) were seeded at ratios of 80:1, 40:1, 20:1, 10:1 and 5:1, and each ratio was prepared in 3 repeats. The co-culture was conducted at 37° C., 5% CO₂ for 24 hours. Post co-culture, the 96-well plate was withdrawn from the incubator and subject to measurement of absorbance at OD535-OD485.
(6) Results analysis: the concentrations of the hIL-2 standard sample and the mhIL-2 test sample were plotted on the x-axis, and the cytotoxicity percentages were plotted on the y-axis so as to evaluate cytotoxicity of the immune cells against tumor cells in activation of the hIL-2 standard sample and the mhIL-2 test sample.

The cytotoxicity assay results can be found in Table 5. At cell ratio of 80:1, cytotoxicity of comparative example 1-activated immune cells against tumor cells was 71% after 4-day co-culture. In contrast, cytotoxicity of experimental example 2-activated immune cells reached 95%. At cell ratio of 40:1, cytotoxicity of comparative example 1-activated immune cells was 27%, while cytotoxicity of experimental example 2-activated immune cells remained 61%. At cell ratio of 20:1, cytotoxicity of comparative example 1-activated immune cells dropped to 13%, while cytotoxicity of experimental example 2-activated immune cells remained 26%. The cytotoxicity assay demonstrated that as the additional disulfide bond between IL-2Rα binding domain and non-IL-2Rα binding domain provided experimental example 2 with higher thermostability, the immune cells could be continuously activated with a 4-day interval as the addition cycle, and cytotoxic activity against tumor cells was persistently maintained.

What is claimed is:

1. A polypeptide, comprising:
a first structural domain, comprising an amino acid sequence with at least 80%, 85%, 90%, 95% or 99% similarity to an amino acid sequence at position 1 to position 34 of SEQ ID NO: 1;
a second structural domain, configured at C-terminus of the first structural domain, comprising an amino acid sequence with at least 80%, 85%, 90%, 95% or 99% similarity to an amino acid sequence at position 35 to position 72 of SEQ ID NO: 1; and
a third structural domain, configured at C-terminus of the second structural domain, comprising an amino acid sequence with at least 80%, 85%, 90%, 95% or 99% similarity to an amino acid sequence at position 73 to position 133 of SEQ ID NO: 1, and being introduced of a first mutation by a substitution of a first amino acid residue with a cysteine residue, wherein:
the polypeptide is introduced of a second mutation by a substitution of a second amino acid residue with another cysteine residue, and the second mutation optionally happens in the first structural domain, the second structural domain or the third structural domain, thereby forming a disulfide bond between the cysteine residue introduced by the first mutation and the cysteine residue introduced by the second mutation.

2. The polypeptide according to claim 1, wherein the first amino acid residue comprises a asparagine residue (Asn, N) at position 77, an aspartate residue (Asp, D) at position 84, a isoleucine residue (Ile, I) at position 92, Lysine residue (Lys, K) at position 97, a threonine residue (Thr, T) at position 111, a phenylalanine residue (Phe, F) at position 117 or a leucine residue (Leu, L) at position 132;
the second amino acid residue comprises a glutamine residue (Gln, Q) at position 11, a leucine residue (Leu, L) at position 17, a leucine residue (Leu, L) at position 18, a tyrosine residue (Tyr, Y) at position 31, a lysine residue (Lys, K) at position 43, a phenylalanine residue (Phe, F) at position 44, a leucine residue (Leu, L) at position 56 or a leucine residue (Leu, L) at position 80.

3. The polypeptide according to claim 1, wherein in C-terminus downstream area of the first amino acid residue, the third structural domain further comprises a third mutation introduced by a substitution of a third amino acid residue with a hydrophilic amino acid residue or a hydrophobic amino acid residue.

4. The polypeptide according to claim 3, wherein the third amino acid residue is a cysteine residue.

5. The polypeptide according to claim 3, wherein the third mutation comprising any one of the followings:
a substitution of the cysteine residue at position 125 with a serine residue (Ser, S);
a substitution of the cysteine residue at position 125 with an alanine residue (Ala, A);
a substitution of the cysteine residue at position 125 with a leucine residue (Leu, L).

6. The polypeptide according to claim 1, wherein the first amino acid residue comprises a threonine residue (Thr, T) at position 111, and the second amino acid residue comprises a lysine residue (Lys, K) at position 43.

7. The polypeptide according to claim 6, wherein in C-terminus downstream area of the first amino acid residue, the third structural domain further comprises a third mutation introduced by a substitution of a third amino acid residue with a hydrophilic amino acid residue or a hydrophobic amino acid residue, and wherein the third amino acid residue is a cysteine residue, and the third mutation comprising any one of the followings:
a substitution of the cysteine residue with a serine residue (Ser, S);
a substitution of the cysteine residue with an alanine residue (Ala, A);
a substitution of the cysteine residue with a leucine residue (Leu, L).

8. The polypeptide according to claim 1, wherein the first structural domain comprises an amino acid sequence identical to any one of amino acid sequences of SEQ ID NOs: 2 to 6; the second structural domain comprises an amino acid sequence identical to any one of amino acid sequences of SEQ ID NOs: 7 to 10; the third structural domain an amino acid sequence identical to any one of amino acid sequences of SEQ ID NOS: 11 to 35.

9. The polypeptide according to claim 1, comprising an amino acid sequence with at least 80%, 85%, 90%, 95% or 99% similarity to any one of amino acid sequences of SEQ ID NOs: 36 to 57.

10. A medicine composition, comprising a polypeptide according to claim 1 and a pharmaceutically acceptable carrier.

11. The medicine composition according to claim 10, wherein the first amino acid residue comprises an asparagine residue (Asn, N) at position 77, an aspartate residue (Asp, D) at position 84, a isoleucine residue (Ile, I) at position 92, a lysine residue (Lys, K) at position 97, a threonine residue (Thr, T) at position 111, a phenylalanine residue (Phe, F) at position 117 or a leucine residue (Leu, L) at position 132;
the second amino acid residue comprises a glutamine residue (Gln, Q) at position 11, a leucine residue (Leu, L) at position 17, a leucine residue (Leu, L) at position 18, a tyrosine residue (Tyr, Y) at position 31, a lysine residue (Lys, K) at position 43, a phenylalanine residue (Phe, F) at position 44, a leucine residue (Leu, L) at position 56 or a leucine residue (Leu, L) at position 80.

12. The medicine composition according to claim 10, wherein in C-terminus downstream area of the first amino acid residue, the third structural domain further comprises a third mutation introduced by a substitution of a third amino acid residue with a hydrophilic amino acid residue or a hydrophobic amino acid residue.

13. The medicine composition according to claim 12, wherein the third amino acid residue is a cysteine residue.

14. The medicine composition according to claim 12, wherein the third mutation comprises any one of the followings:
a substitution of the cysteine residue at position 125 with a serine residue (Ser, S);
a substitution of the cysteine residue at position 125 with an alanine residue (Ala, A);
a substitution of the cysteine residue at position 125 with a leucine residue (Leu, L).

15. The medicine composition according to claim 1, wherein the first structural domain comprises an amino acid sequence identical to any one of amino acid sequences of SEQ ID NOs: 2 to 6; the second structural domain comprises an amino acid sequence identical to any one of amino acid sequences of SEQ ID NOs: 7 to 10; the third structural domain an amino acid sequence identical to any one of amino acid sequences of SEQ ID NOs: 11 to 35.

16. The medicine composition according to claim 1, comprising an amino acid sequence with at least 80%, 85%, 90%, 95% or 99% similarity to any one of amino acid sequences of SEQ ID NOs: 36 to 57.

17. A use of the medicine composition according to claim 10, wherein the medicine composition used for preparing a medical or cell therapeutic composition to treat, prevent or relieve a disease or a disorder caused by diminished immune cell activity.

18. The use according to claim 17, wherein the immune cell comprises natural killer cells, NK-T cells, T cells, B cells, macrophages, dendritic cells, oligodendrocyte, cytokine Induced Killer cells, or γδT cells.

19. The use according to claim 17, wherein the disease comprises a proliferative disorder including hematologic malignancy, solid tumors or metastatic tumors.

20. The use according to claim 17, wherein:
the first amino acid residue comprises a asparagine residue (Asn, N) at position 77, a aspartate residue (Asp, D) at position 84, an isoleucine residue (Ile, I) at position 92, a lysine residue (Lys, K) at position 97, a threonine residue (Thr, T) at position 111, a phenylalanine residue (Phe, F) at position 117 or a leucine residue (Leu, L) at position 132;
the second amino acid residue comprises a glutamine residue (Gln, Q) at position 11, a leucine residue (Leu, L) at position 17, a leucine residue (Leu, L) at position 18, a tyrosine residue (Tyr, Y) at position 31, a lysine residue (Lys, K) at position 43, a phenylalanine residue (Phe, F) at position 44, a leucine residue (Leu, L) at position 56 or a leucine residue (Leu, L) at position 80;
in C-terminus downstream area of the first amino acid residue, the third structural domain further comprises a third mutation introduced by a substitution of a third amino acid residue with a hydrophilic amino acid residue or a hydrophobic amino acid residue, and the third mutation comprises a substitution of a cysteine residue at position 125 with a serine residue (Ser, S), an alanine residue (Ala, A), or a leucine residue (Leu, L).
